(19) United States
(12) Patent Application Publication
(10) Pub. No.: US 2012/0078141 A1
(43) Pub. Date: Mar. 29, 2012

(54) Systems and methods for using reverse thermal gradient to non-invasively heat a subjacent soft tissue structure

(76) Inventor: Edward W. Knowlton, Zehpyr Cove, NV (US)

(21) Appl. No.: 13/218,595

(22) Filed: Aug. 26, 2011

Related U.S. Application Data
(60) Provisional application No. 61/380,049, filed on Sep. 3, 2010.

(51) Int. Cl. A61N 5/00; A61N 5/06; A61B 18/02; A61N 7/02; A61N 1/28; A61B 18/18; A61N 5/02; A61N 5/067

(52) U.S. Cl. 601/3; 607/100; 607/101; 607/90; 607/89; 607/99; 606/33; 606/20

(57) ABSTRACT

A new non-invasive approach is proposed that contemplates a method and apparatus to utilize two- or three-dimensional treatment patterns with a reverse thermal gradient to non-invasively heat a subjacent soft tissue structure through an intact tissue surface or an intact surface epithelium for clinical applications. For most clinical applications, an electromagnetic energy source with surface cooling is employed to heat the treatment patterns. Without limitation, the clinical applications include but are not limited to the treatment of post partum vaginal laxity, female incontinence, cervical incompetence with preterm labor, gastro-esophageal reflux, reduction of gastric reservoir capacity (for weight management), sleep apnea, snoring, pain management and the treatment of orthopedic injuries such as joint laxity and tennis elbow.

### RELATED APPLICATIONS

This application claims the benefit of U.S. Provisional Patent Application No. 61/380,049, filed Sep. 3, 2010, and entitled “Systems and methods for using reverse thermal gradient to non-invasively heat a subjacent soft tissue structure,” by Edward W. Knowlton, and is hereby incorporated herein by reference.

### BACKGROUND

Reverse thermal technology provides several potential benefits for the treatment of common disease states. The principle benefits include the ability to non-invasively treat patients without surgery and to perform as needed, a sequence of repeated treatments with a minimal recovery period. The thermal induction of the delayed wound healing serves as the primary mechanism of action for this treatment process. This biological process is initiated by the thermal denaturation and biophysical contraction of the pre-existing collagen matrix. Within 3-4 days of the initial thermal injury, a period of fibroplasia develops in which the preexisting denatured collagen matrix is replaced with nascent collagen deposition. During this phase of collagen deposition, the treated matrix is secondarily tightened with process called cellular myofibroblastic contraction.

Furthermore, thermally denatured treatment areas that are devoid of native collagen will also be subject to scar collagen deposition and delayed tightening during this phase of fibroplasia. Although surgical corrective procedures share the same wound healing mechanism of action, the invasive nature of surgery with its obligate scarring has served as a stimulus to the development of reverse thermal technology. The clinical applications of this non-invasive approach are being employed in several areas that are currently being managed by surgery.

The foregoing examples of the related art and limitations related therewith are intended to be illustrative and not exclusive. Other limitations of the related art will become apparent upon a reading of the specification and a study of the drawings.

### BRIEF DESCRIPTION OF THE DRAWINGS

FIG. 1 depicts an example of a two-dimensional treatment pattern for a two-dimensional clinical application of wound healing response.

FIG. 2 depicts an example of a three-dimensional treatment pattern for a directed three-dimensional wound healing response on a tube.

FIG. 3 depicts an example of a three-dimensional treatment pattern for post-partum vaginal laxity.

FIGS. 4(a)-(b) depict examples of curved three-dimensional circular and semi-spherical treatment patterns used to tighten a three-dimensional tubular structure.

FIG. 5 depicts an example of a system of three-dimensional treatment patterns used in combination with a system of non-contiguous two-dimensional treatment patterns.

FIG. 6 depicts an example of a directed three-dimensional wound healing response used for a clinical application on a soft tissue structure covered with skin.

FIG. 7 depicts an example of a three-dimensional aesthetic contouring with a superficial two-dimensional treatment pattern tightens the skin and a deep two-dimensional treatment pattern achieves inward contouring by thermal lipolysis of the subcutaneous fat layer.

FIG. 8 depicts an example of an apparatus embodiment that mitigates residual electrode edge effect via direct coupling.

FIG. 9 depicts an example of an apparatus embodiment that mitigates residual electrode edge effect via capacitive coupling.

FIG. 10 depicts an example of an apparatus embodiment that mitigates residual electrode edge effect via capacitively coupled treatment tip with a “framed” dielectric membrane.

FIG. 11 depicts an example of an apparatus embodiment that mitigates residual electrode edge effect via capacitively coupled treatment tip with a progressively “doped” dielectric membrane.

FIG. 12 depicts an example of an apparatus embodiment that mitigates residual electrode edge effect via capacitively coupled treatment tip with a combination of both framed and progressively doped dielectric membrane.

FIG. 13 depicts an example of an apparatus embodiment that mitigates residual electrode edge effect via a plurality of concentric tip electrodes in a single treatment tip.

FIG. 14 depicts an example of an apparatus embodiment that mitigates residual electrode edge effect via a RF system having directly coupled treatment tip with a perimeter “skirt” cooling component.

FIG. 15 depicts an example of an apparatus embodiment that mitigates residual electrode edge effect via gradual doping or concentric layering to alter the electrical resistance/thermal conductivity of the perimeter of the electrode.

FIG. 16 depicts an example of an apparatus embodiment that mitigates residual electrode edge effect via a curved dielectric electrode surface to diminish both electrode and pressure edge effects.

FIG. 17 depicts an example of a two bar bipolar electrode to create an electrical field that has the greatest field density in a longitudinal pattern between and parallel to the two bipolar bar electrodes.

### DETAILED DESCRIPTION OF EMBODIMENTS

The approach is illustrated by way of example and not by way of limitation in the figures of the accompanying drawings in which like references indicate similar elements. It should be noted that references to “an” or “one” or “some” embodiment(s) in this disclosure are not necessarily to the same embodiment, and such references mean at least one.

A new non-invasive approach is proposed that contemplates a method and apparatus to utilize two- or three-dimensional treatment patterns with a reverse thermal gradient to non-invasively heat a subjacent soft tissue structure through an intact tissue surface or an intact surface epithelium for clinical applications. For most clinical applications, an RF energy source with surface cooling is employed to heat the treatment patterns. However, other electromagnetic energy sources such as optical, laser, ultrasound, microwave and resistive heating may also be employed. Without limitation, the clinical applications include but are not limited to the treatment of post partum vaginal laxity, female incontinence, cervical incompetence with preterm labor, gastro-esophageal reflux, reduction of gastric reservoir capacity (for weight management), sleep apnea, snoring, pain management and the treatment of orthopedic injuries such as joint laxity and tennis elbow.

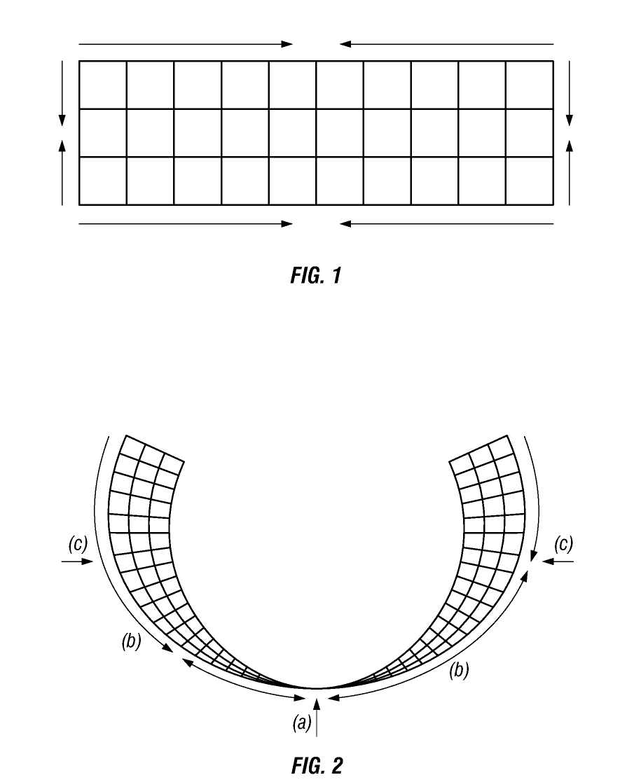

FIG. 1 depicts an example of a two-dimensional clinical application of wound healing response, which can be but is not limited to a surface tightening of lax skin, under a two-dimensional treatment pattern to non-invasively heat a subjacent soft tissue structure through an intact tissue surface or an intact surface epithelium using an electromagnetic energy source with surface cooling. Here, the lengths of the arrows indicate strengths of forces of the directed wound healing response and the directions of the arrows indicate the directions of the wound healing response.

In an alternative embodiment, a directed (“vectored”) three-dimensional wound healing response from a three-dimensional treatment pattern can be employed for the treatment of additional clinical applications on, for a non-limiting example, a tube, as shown by the example depicted in FIG. 2. As shown in FIG. 2, the major effect (a) and (b) of applying the three-dimensional application pattern is to raise or tighten diameter of the tube while the minor effect (c) of applying the three-dimensional application pattern is to shorten the length of the tube.

In another preferred embodiment, the three-dimensional treatment pattern and the vectored three-dimensional soft tissue wound healing response of a specific clinical application are virtually designed and configured with a software program prior to treatment. In addition to simulating a thermal lesion with the appropriate orientation in a virtual three-dimensional space, the software (and its database) also takes into consideration descriptions of one or more of the thermal dosimetry, the dimensions and the depth of the thermal lesion required for an optimal treatment.

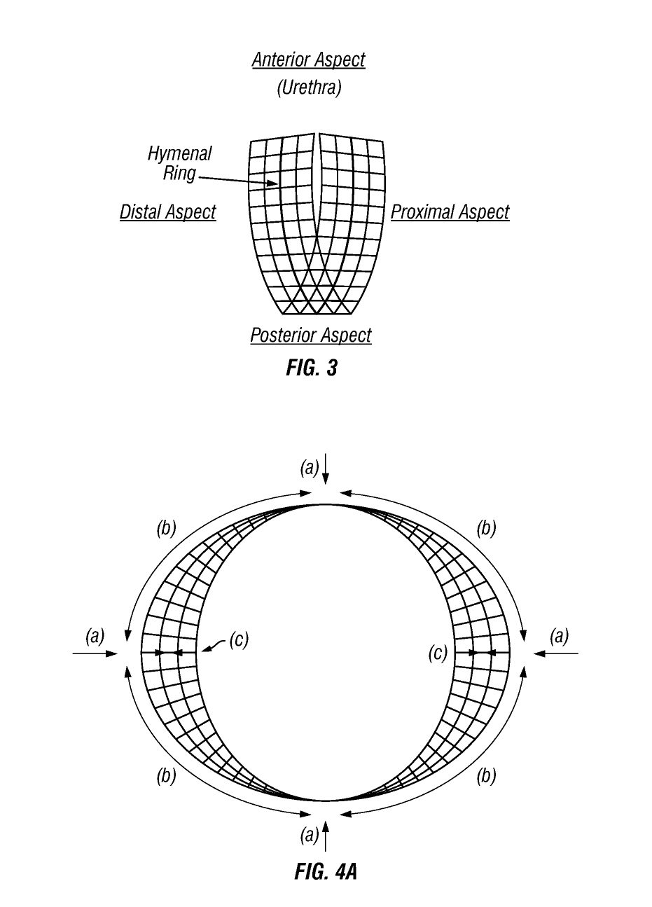

FIG. 3 depicts an example of a three-dimensional treatment pattern for clinical application for treatment of post-partum vaginal laxity. As shown in FIG. 3, a curved three-dimensional wound healing response is adopted for a clinical application inside a tubular anatomical structure lined with mucosa. Here, the curved three-dimensional wound healing response includes a posterior aspect, a proximal aspect, a distal aspect with a hymenal ring, and an anterior aspect. The use the curved three-dimensional semicircular and circular treatment patterns tighten a three-dimensional tubular structure such as the vagina, the cervix and the female urethra as shown in FIG. 3.

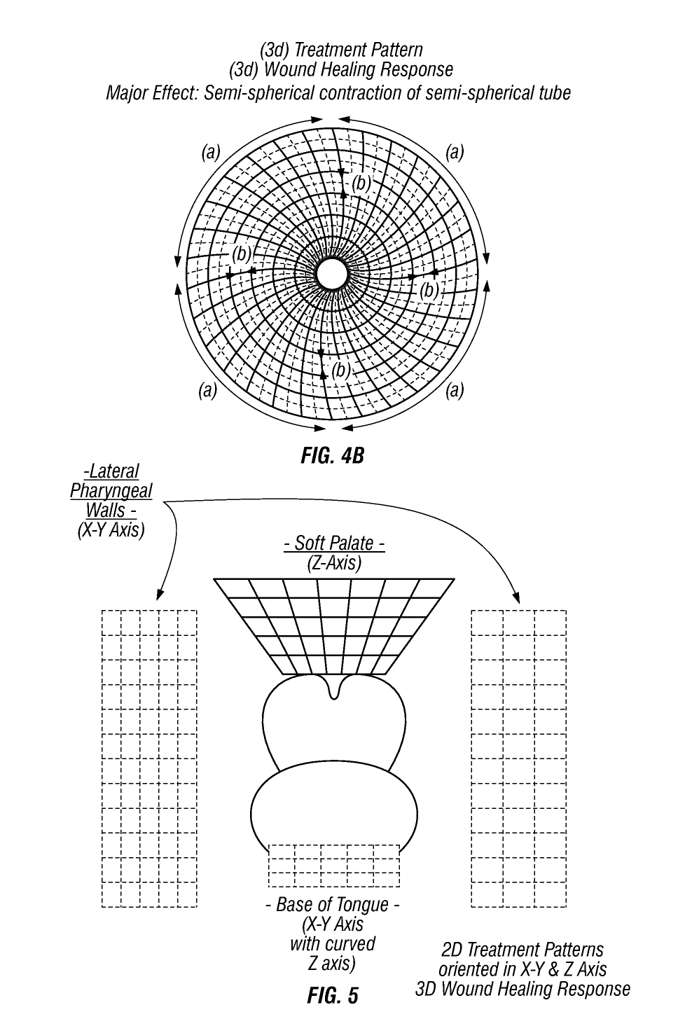

In some embodiments, a curved three-dimensional circular and semi-spherical treatment pattern can be used to tighten a three-dimensional tubular structure such as a gastrointestinal tract including gastro-esophageal junction, stomach and the rectal sphincter as shown by the examples depicted in FIGS. 4(a) and 4(b). In FIG. 4(a), the major effect (a) and (b) of applying the three-dimensional circular treatment pattern is to circumference or tighten the tube while the minor effect (c) of applying the three-dimensional circular treatment pattern is to shorten the length of the tube. In FIG. 4(b), the major effect (a) and (b) of applying the three-dimensional semi-spherical treatment pattern is to cause contraction of the semi-spherical tube.

In some embodiments, a system of three-dimensional treatment patterns can be used to maintain the diameter of a three-dimensional tubular structure such as the oropharynx in combination with a system of non-contiguous two-dimensional treatment patterns used to maintain the patency of the upper airway as shown by the example depicted in FIG. 5. As shown in FIG. 5, the two-dimensional treatment patterns are oriented in different axis to each other with respect to the three-dimensional treatment patterns to stent (splint) the soft palate (z axis), lateral pharyngeal walls (x-y axis) and the base of the tongue (x-y axis with curved z axis).

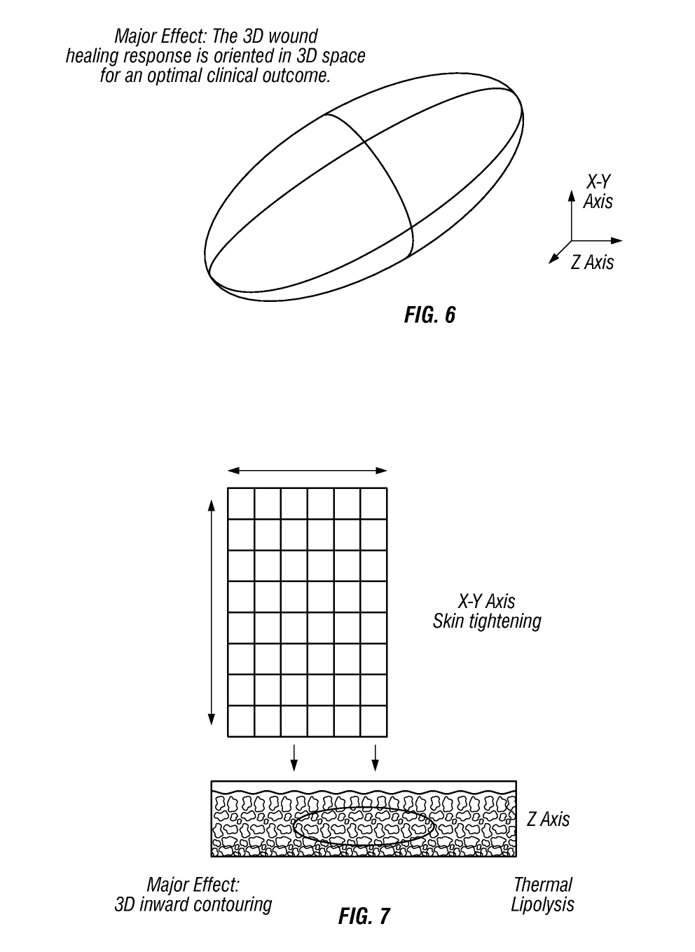

In some embodiments, a directed (vectored) three-dimensional wound healing response can be used for a clinical application on a soft tissue structure covered with skin as shown by the example depicted in FIG. 6, where the major effect of the three-dimensional wound healing response is oriented in a three-dimensional space for an optimal clinical outcome.

In some embodiments, a three-dimensional aesthetic contouring is achieved with a superficial two-dimensional treatment pattern that tightens the skin and a deep two-dimensional treatment pattern that achieves inward contouring by thermal lipolysis of the subcutaneous fat layer as shown by the example depicted in FIG. 7. In other embodiments, a three-dimensional semicircular treatment pattern of the neck skin can be used to raise a three-dimensional structure of the neckline as shown by the example depicted in FIG. 2.

In some embodiments, the clinical applications are functional orthopedic applications that create an overall three-dimensional wound healing response involving one or more of: a) two-dimensional tightening of a collagen-containing surface, and b) a z axis (depth) thickening of the orthopedic structure (for non-limiting examples, fascia, tendon and ligaments). The three-dimensional wound healing response is also vectored to enhance the mechanical function of the orthopedic structure in three-dimensional space of the body.

In some embodiments, the clinical applications achieve a three-dimensional modification of a physiological process, such as nerve function in which cutaneous pain receptors are suppressed over a broader two-dimensional skin surface area, and/or deeper structures such as muscles, which are suppressed with a deeper and more focused two-dimensional application pattern.

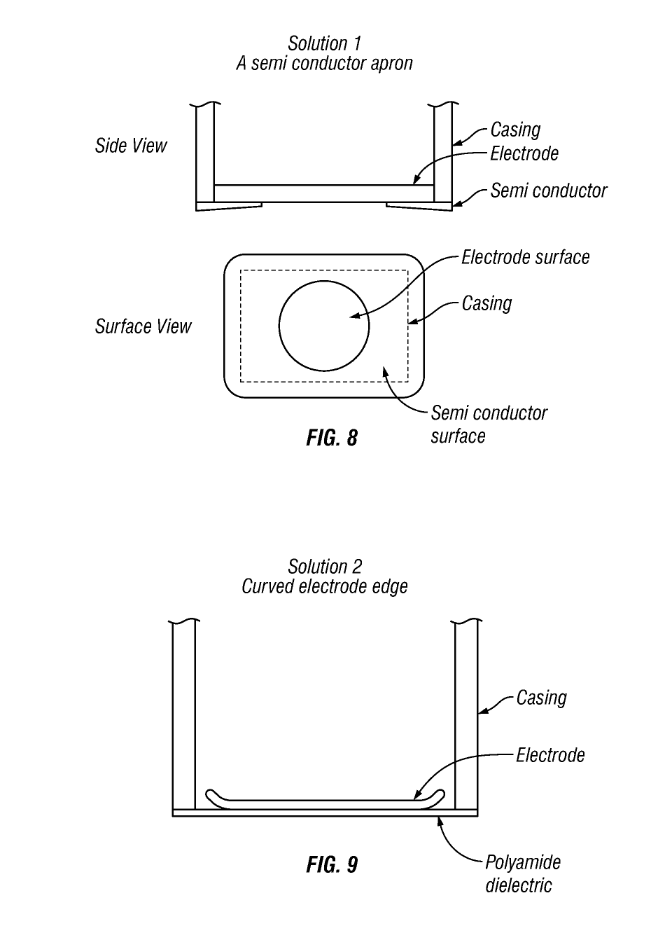

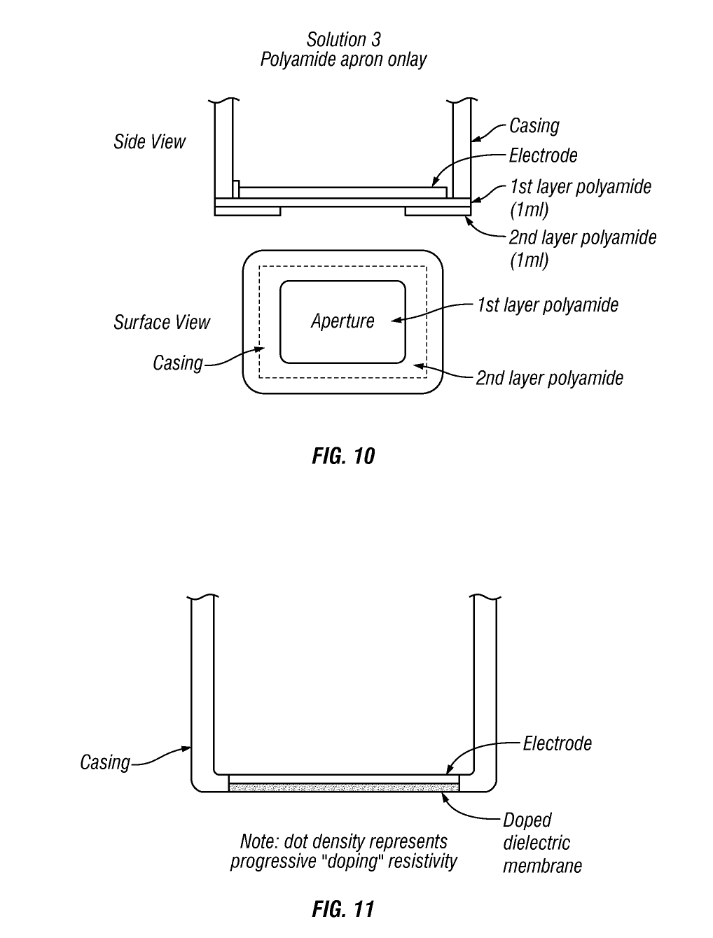

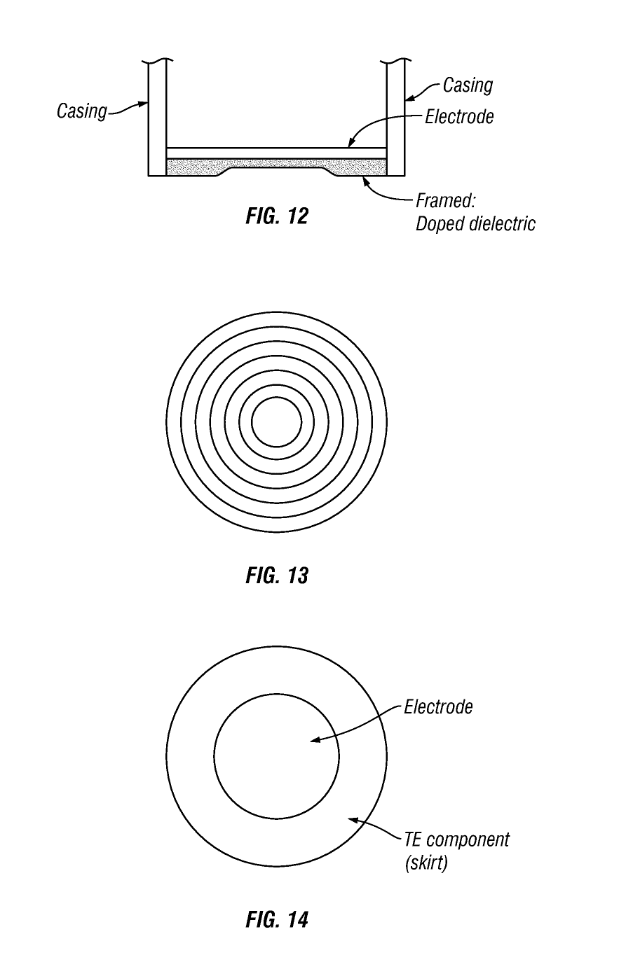

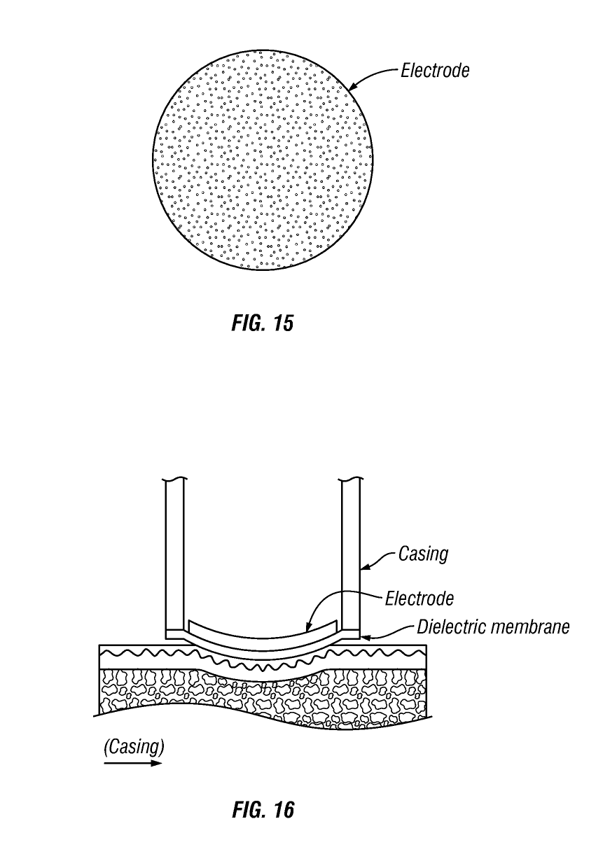

In some embodiments, an application tip (electrode) is required to create a uniform three-dimensional thermal lesion and a directed (vectored) three-dimensional wound healing response in order to achieve an optimal clinical outcome. For this reason, any residual electrode edge effect of the application tip must be mitigated for charge dissipation of the electrode edge with the establishment of even charge distribution over the electrode surface. The following are non-limiting examples of apparatus embodiments that mitigate residual electrode edge effect:
Direct coupling: a semiconductor apron that covers the electrode edge is depicted in the example of FIG. 8. A shown in FIG. 8, a central (circular or square) aperture in the semiconductor apron allows direct contact/coupling between the central aspect of the electrode and the skin surface. To evenly distribute the charge, the thickness of the semiconductor apron is progressively increased towards the electrode edge i.e., the apron is thinnest at the perimeter of the aperture and thickest under the electrode edge. Such device reduces power required and enhances cooling efficiency of the device.
Capacitive coupling: the electrode is capacitively coupled to the skin surface via a dielectric membrane where the electrode edge is curved away from contact with the dielectric membrane as depicted in the example of FIG. 9. Here, the extent and slope of electrode edge curving can be modified to maximally reduce electrode edge effect without affecting power requirements and cooling efficiency of the device.
Capacitive coupled RF system that includes one or more of:
   A capacitively coupled treatment tip (electrode edge) with a dielectric membrane having a thickened “framed” dielectric at the perimeter of the tip, which side view and surface view are depicted in the example of FIG. 10. The progressively increasing thickness of polyamide from the aperture is subjacent to the electrode edge and a single thickness of dielectric covers the central portion of the electrode. In FIG. 10, a dielectric (1 ml polyamide) apron with a central (circular or square) aperture is applied as an onlay over multiple layers of 1 ml polyamide membrane.
   A progressively “doped” dielectric membrane that becomes more resistive towards the perimeter of the tip as depicted in the example of FIG. 11. In FIG. 11, the dot density represents progressive “doping” and resistivity of the doped dielectric. Another embodiment involves a semiconductor membrane that is layered over the dielectric membrane and is comprised of a thin central portion and is of increasing thickness towards the casing. An even redistribution of charge is achieved across the composite dielectric and semiconductor membrane.
   A combination of both framed and progressively doped dielectric membrane as depicted in the example of FIG. 12.
   A plurality of concentric tip electrodes in a single application/treatment tip as depicted in the example of FIG. 13.
An RF system having the application tip directly coupled (without a dielectric membrane) with a perimeter “skirt” cooling component as depicted in the example of FIG. 14. Alternatively, an RF system having the application of a capacitively coupled tip with a dielectric membrane with a perimeter “skirt” cooling component adjacent to the RF electrode can be adopted. Here, one or more of following cooling mechanisms can be adopted: thermoelectric cooling, fluidic cooling, and phase transition with a cryogen spray fluid. In some embodiments, gradual doping or concentric layering (with different materials) to alter the electrical resistance/thermal conductivity of the perimeter of the electrode can be adopted to further reduce electrode edge effect as depicted in the example of FIG. 15 where dot density represents progressive doping and resistivity.
A curved dielectric electrode surface to diminish both electrode and pressure edge effects as depicted in the example of FIG. 16. Avoidance of “edge effects” is primarily achieved by uniformly distributing the electrical charge over an electrode surface. However, the application of excessive pressure will contribute secondarily to an electrode edge effect. Mechanical displacement of extra cellular fluid (ECF) will increase local impedance of the compressed tissue. In a uniform electrical field, the compressed and more resistive tissue will be heated more than non-compressed tissue. Another effect of pressure is to bring subjacent structures into the electrical field. This combined electrical pressure edge effect is accentuated over a bony prominence. In this circumstance, the avoidance of electrical and pressure edge effects is important to the avoidance of surface burning and the avoidance of subjacent neurovascular structures. In FIG. 16, the electrode edge and pressure (casing) edge do not contact the skin and the charge and pressure are evenly distributed over the skin treatment area.

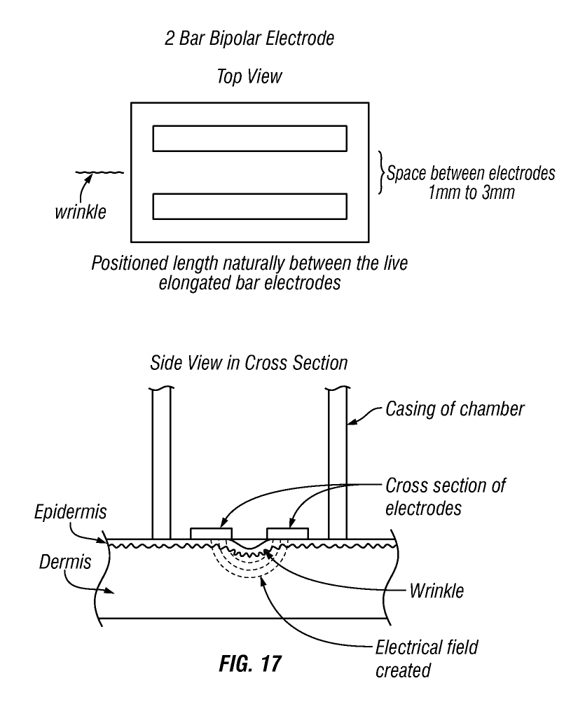

In some embodiments, a two-bar bipolar electrode as depicted in the example of FIG. 17 can be used, where the current density is greatest between the two electrodes in a bipolar electrical field. A parallel pair of bar shaped of electrodes will create an electrical field that has the greatest field density in a longitudinal pattern between and parallel to the two bipolar bar electrodes. The increased field density in a longitudinal pattern has a specific application for correction of longitudinal dermal defects such as wrinkles and striae. For wrinkles, the depressed skin contour will be corrected by an immediate (denaturation) contraction of native collagen in the cleft of the wrinkle. The raised skin contour is subsequently supported by a wound healing sequence that deposits scar collagen in the thermal contracted (denatured) collagen matrix. The double bar electrode can be deployed with or without a dielectric membrane as described in electrode embodiments discussed above. For striae, the dermal defect is not from aging (where a gradual loss of the dermal matrix occurs) but from a tearing or mechanical disruption of the supporting dermal matrix of the skin. Striae occur most frequently on the lower abdomen of pregnant women. The application of the double bar electrode on each side of the striae will reduce the width of this longitudinally oriented defect. Another application of the double bar electrode is the treatment of sphincter incontinence. In this case the electrode is applied circumferentially around the mucosa (transmucosally) of a physiological sphincter. The circumferential raised contour of the sphincter will circumferentially contract the diameter of the sphincter. Examples of potential sites to be treated are the GE junction, rectal sphincter, proximal female urethra, rectal sphincter and an intestinal stoma.

The foregoing description of various embodiments of the invention has been presented for purposes of illustration and description. Various methods of the invention are applicable to variety of medical, dermatological and surgical methods including reconstructive and plastic surgery procedures and minimally invasive procedures. It is not intended to limit the invention to the precise forms disclosed. Many modifications, variations and different combinations of embodiments will be apparent to practitioners skilled in this art. Further, elements from one embodiment can be readily recombined with one or more elements from other embodiments.

1. An apparatus for treating skin tissue, comprising:
a two-dimensional treatment pattern to create a directed wound healing response to non-invasively heat a subjacent soft tissue structure through an intact tissue surface or an intact surface epithelium for a two-dimensional clinical application; and
an electromagnetic energy source with surface cooling employed for the two-dimensional treatment pattern.

2. The apparatus of claim 1, wherein:
the electromagnetic energy source is an RF energy source.

3. The apparatus of claim 1, wherein:
the electromagnetic energy source is one of optical, laser, ultrasound, microwave and resistive heating.

4. The apparatus of claim 1, wherein:
the two-dimensional clinical applications is treatment of one or more of: post partum vaginal laxity, female incontinence, cervical incompetence with preterm labor, gastro-esophageal reflux, reduction of gastric reservoir capacity, sleep apnea, snoring, pain management and the treatment of orthopedic injuries such as joint laxity and tennis elbow.

5. The apparatus of claim 1, wherein:
the wound healing response is a surface tightening of lax skin.

6. An apparatus for treating skin tissue, comprising:
a three-dimensional treatment pattern to create a directed three-dimensional wound healing response to non-invasively heat a subjacent soft tissue structure through an intact tissue surface or an intact surface epithelium for a three-dimensional clinical application; and
an electromagnetic energy source with surface cooling employed for the three-dimensional treatment pattern.

7. The apparatus of claim 6, wherein:
the clinical application is on a tube and applying the three-dimensional application pattern on the tube raises or tightens diameter of the tube while shortening the length of the tube.

8. The apparatus of claim 6, wherein:
the three-dimensional treatment pattern and the directed three-dimensional soft tissue wound healing response of the specific clinical application are virtually designed and configured with a software program prior to treatment.

9. The apparatus of claim 8, wherein:
the software takes into consideration descriptions of one or more of the thermal dosimetry, the dimensions and the depth of the thermal lesion required for an optimal treatment in addition to simulating a thermal lesion with the appropriate orientation in a virtual three-dimensional space.

10. The apparatus of claim 6, wherein:
the clinical application is treatment of post-partum vaginal laxity where a curved three-dimensional wound healing response is adopted for an application inside a tubular anatomical structure lined with mucosa.

11. The apparatus of claim 6, wherein:
the three-dimensional treatment pattern is a curved three-dimensional semicircular or circular treatment pattern that tightens a three-dimensional tubular structure.

12. The apparatus of claim 6, wherein:
the three-dimensional treatment pattern is a curved three-dimensional circular and semi-spherical treatment pattern that tightens a gastrointestinal tract by circumferencing or tightening the tract while shortening the length of the tract.

13. The apparatus of claim 6, further comprising:
a non-contiguous two-dimensional treatment pattern used to maintain the patency of the upper airway in combination with the three-dimensional treatment pattern used to maintain the diameter of a three-dimensional tubular structure.

14. The apparatus of claim 13, wherein:
the two-dimensional treatment pattern is oriented in different axis to each other with respect to the three-dimensional treatment pattern to stent one or more of soft palate, lateral pharyngeal walls and the base of the tongue.

15. The apparatus of claim 6, wherein:
the clinical application is on a soft tissue structure covered with skin where the three-dimensional wound healing response is oriented in a three-dimensional space for an optimal clinical outcome.

16. The apparatus of claim 6, further comprising:
a superficial two-dimensional treatment pattern that tightens the skin and a deep two-dimensional treatment pattern that achieves inward contouring by thermal lipolysis of the subcutaneous fat layer for a three-dimensional aesthetic contouring.

17. The apparatus of claim 6, further comprising:
a three-dimensional semicircular treatment pattern of the neck skin to raise a three-dimensional structure of the neckline.

18. The apparatus of claim 6, wherein:
the clinical application is a functional orthopedic application that creates an overall three-dimensional wound healing response involving one or more of: two-dimensional tightening of a collagen-containing surface, and a z axis (depth) thickening of the orthopedic structure.

19. The apparatus of claim 18, wherein:
the three-dimensional wound healing response is vectored to enhance the mechanical function of the orthopedic structure in three-dimensional space of the body.

20. The apparatus of claim 6, wherein:
the clinical application achieves a three-dimensional modification of a physiological process in which cutaneous pain receptors are suppressed over a broader two-dimensional skin surface area, or deeper structures which are suppressed with a deeper and more focused two-dimensional application pattern.

21. The apparatus of claim 6, further comprising:
an electrode required as an application tip to create a uniform three-dimensional thermal lesion and the directed three-dimensional wound healing response in order to achieve an optimal clinical outcome.

22. The apparatus of claim 21, further comprising:
an apparatus that mitigates residual edge effect of the application tip for charge dissipation of the electrode with establishment of even charge distribution over surface of the electrode.

23. The apparatus of claim 22, wherein:
the apparatus that mitigates residual edge effect is a semiconductor apron that covers edge of the electrode with a central aperture in the semiconductor apron to allow direct coupling between the central aspect of the electrode and the skin surface.

24. The apparatus of claim 22, wherein:
the electrode is capacitively coupled to the skin surface via a dielectric membrane where the edge of the electrode is curved away from contact with the dielectric membrane and the extent and slope of electrode edge curving is modified to maximally reduce electrode edge effect.

25. The apparatus of claim 24, wherein:
the dielectric membrane has a thickened “framed” dielectric at the perimeter of the application tip.

26. The apparatus of claim 25, wherein:
the progressively increasing thickness of polyamide from the aperture is subjacent to the electrode edge and a single thickness of dielectric covers the central portion of the electrode.

27. The apparatus of claim 24, wherein:
the dielectric membrane is progressively doped to become more resistive towards the perimeter of the application tip.

28. The apparatus of claim 24, further comprising:
a semiconductor membrane that is layered over the dielectric membrane and is comprised of a thin central portion and is of increasing thickness towards the casing to achieve an even redistribution of charge across the composite dielectric and semiconductor membrane.

29. The apparatus of claim 24, wherein:
the dielectric membrane has a thickened “framed” dielectric at the perimeter of the application tip and is progressively doped to become more resistive towards the perimeter of the application tip.

30. The apparatus of claim 22, wherein:
the apparatus that mitigates residual edge effect is an RF system having the application of a directly coupled tip with a perimeter “skirt” cooling component without a dielectric membrane.

31. The apparatus of claim 22, wherein:
the apparatus that mitigates residual edge effect is an RF system having the application of a capacitively coupled tip with a dielectric membrane with a perimeter “skirt” cooling component adjacent to the RF electrode.

32. The apparatus of claim 30, wherein:
the cooling component adopts one or more of thermoelectric cooling, fluidic cooling, and phase transition with a cryogen spray fluid.

33. The apparatus of claim 22, wherein:
the apparatus that mitigates residual edge effect includes gradual doping or concentric layering with different materials to alter the electrical resistance/thermal conductivity of the perimeter of the electrode.

34. The apparatus of claim 22, wherein:
the apparatus that mitigates residual edge effect includes a curved dielectric surface of the electrode to diminish both electrode and pressure edge effects by uniformly distributing the electrical charge over the electrode surface.

35. The apparatus of claim 6, further comprising:
a two-bar bipolar electrode comprising a parallel pair of bar shaped of electrodes to create an electrical field that has the greatest field density in a longitudinal pattern between and parallel to the two bipolar bar electrodes, wherein the increased field density in a longitudinal pattern has a specific application for correction of longitudinal dermal defects.

36. The apparatus of claim 35, wherein:
the longitudinal dermal defects corrected include wrinkles and striae.
